(19) United States
(12) Patent Application Publication
(10) Pub. No.: US 2011/0301135 A1
(43) Pub. Date: Dec. 8, 2011

(54) STABILIZATION OF GLUCOCORTICOID ESTERS WITH ACIDS

(75) Inventors: DIRK MERTIN, Langenfeld (DE); Iris Heep, Koeln (DE); Georg Schulte, Wuppertal (DE); Ulrike Umgelder, Leverkusen (DE); Gert Daube, Engelskirchen (DE); Ernst B+e,uml o+ee ttcher, Koeln (DE)

(73) Assignee: BAYER ANIMAL HEALTH GMBH, Leverkusen (DE)

(21) Appl. No.: 12/954,989

(22) Filed: Nov. 29, 2010

Related U.S. Application Data
(62) Division of application No. 11/721,209, filed on Jun. 8, 2007, now abandoned.

(30) Foreign Application Priority Data
Dec. 9, 2004 (DE) 10 2004 059 220.9
Nov. 17, 2005 (DE) 10 2005 055 385.0
Nov. 17, 2005 (DE) 10 2005 055 386.9

(51) Int. Cl. A61K 31/573; A61K 31/56

(52) U.S. Cl. 514/180; 514/169

(57) ABSTRACT

The invention relates to nonaqueous pharmaceutical preparations comprising a glucocorticoid ester and an acid, and to the stabilization of glucocorticoid esters in such preparations by acids.

The invention relates to nonaqueous pharmaceutical preparations comprising a glucocorticoid ester and an acid, and to the stabilization of glucocorticoid esters in such preparations by acids.

Since it has been possible to prepare glucocorticoids by synthesis they have been employed for the treatment of inflammatory disorders in human and veterinary medicine. However, on long-term systemic administration there is frequently, owing to the rising corticoid level in the blood, development of so-called Cushing's syndrome with moon face, steroid acne, central obesity, plethora, stretch marks on the skin (striae rubrae), essential hypertension, general deficiency in vitality, endocrine psychosyndrome, osteoporosis, diabetes mellitus, impotence, oligo- to amenorrhoea, hypertrichosis and hirsutism. In addition, the risk of infections and the flaring up of latent infections is increased, gastric ulcers may be activated, and wound healing is delayed. Because of the catabolic effect, atrophies of muscles, skin and adipose tissue are possible. The risk of thrombosis is increased.

In order to keep the systemic exposure to glucocorticoids low, attempts are made to bring the active ingredient directly to the site of the disorder by topical application. In this case, only about 1-10% of the applied dose is systemically available. Inflammations of the skin are usually treated by local application of semisolid (ointments, creams, gels) or liquid pharmaceutical forms (suspensions, emulsions, solutions) in which a glucocorticoid is dissolved or dispersed.

Besides glucocorticoids, also glucocorticoid esters are known.

Esterification of the hydroxyl groups at C17 and/or C21 increases the potency of the glucocorticoids. The greater lipophilicity leads to better penetration into cells. At the same time, accumulation in the skin is improved. Thus, for example, hydrocortisone is one of the weak glucocorticoids, whereas hydrocortisone 17-butyrate is one of the strong glucocorticoids. Similar effects are to be expected with the glucocorticoids dexamethasone—dexamethasone 21-acetate and betamethasone—betamethasone 17-valerate.

However, glucocorticoid esters are more or less sensitive to hydrolysis, being converted into the corresponding less active, unesterified corticoids. This hydrolysis by its nature takes place especially in the abovementioned topical pharmaceutical forms when in an aqueous formulation. However, hydrolysis cannot be completely precluded even in anhydrous formulations because of uptake of moisture from the surroundings. The use of packagings impermeable to water vapour often fails from aesthetic or economic considerations.

However, it is possible to stabilize the corticoid esters by adjusting the pH into the slightly acidic range. Hydrolysis is reduced there by comparison with the more strongly acidic and neutral-basic pH range (Anderson B D et al, Strategies in the design of solution-stable, water-soluble prodrugs I: a physical-organic approach to pro-moiety selection of 21-esters of corticosteroids, J. Pharm. Sci. 74(4), 365-374, 1985; Gonzalo-Lumbreras R et al., High-performance liquid chromatographic separation of corticoid alcohols and their derivatives: a hydrolysis study including application to pharmaceuticals, J. Chromatogr. Sci. 35(9), 439-445, 1997).

Powder mixtures containing corticoid esters have also been stabilized by adding organic acids (Teijin Ltd., Powdery pharmaceuticals, for treatment of oral cavity disorders, containing steriodal inflammation inhibitors and organic acids stabilizers, JP60028923; Teijin Ltd., Powder compositions containing beclomethasone dipropionate for nasal mucous membrane application, JP60032714). The described powder formulations contain considerable amounts of water which is introduced via the further excipients (e.g. cellulose ethers). In addition, further water may be taken up from the humidity of the surrounding air. It is thus to be presumed that the pH in the water layer then adhering to the powder particles is shifted by the addition of acid, and thus the corticoid esters are stabilized.

A shift in the pH by adding organic or inorganic acids is, however, by its nature possible only in the case of the aforementioned aqueous or water-containing preparations. It has now surprisingly been found that addition of acids to nonaqueous dissolving or dispersing media can likewise stabilize the glucocorticoid esters to hydrolysis, although the acids cannot dissociate in these dissolving or dispersing media.

The invention therefore relates to nonaqueous, fluid pharmaceutical preparations comprising at least one glucocorticoid ester and at least one acid.

Glucocorticoid esters are normally esters of the glucocorticoids with organic acids such as, for example, carboxylic acids or carbonic acid compounds. The hydroxyl group at C17 or C21 of the corticoid is preferably esterified, but esterification of both hydroxyl groups is also possible. The acid component of the ester is derived for example from saturated aliphatic carboxylic acids having up to 10, preferably up to 8, particularly preferably up to 6, carbon atoms. Examples of such esters which may be mentioned are: acetates, propionates, butyrates, valerates, hexanoates, pivalates.

Aceponate refers to a mixed propionate-acetate diester, and buteprate refers to a mixed butyrate-acetate diester. Further suitable esters are derived from heterocyclically substituted carboxylic acids, such as, for example, the furoates. Likewise suitable are mixed carbonic esters resulting from the introduction of an alkoxycarbonyl group, preferably having 1 to 6 carbon atoms; an example which may be mentioned is the ethoxycarbonyl group.

Examples of glucocorticoid esters are aclometasone propionate, betamethasone dipropionate, betamethasone valerate, clobetasol propionate, clobetasone butyrate, clocortolone hexanoate, clocortolone pivalate, dexamethasone acetate, diflucortolone valerate, flumetasone pivalate, fluocortolone hexanoate, fluocortolone pivalate, fluprednidene acetate, fluticasone propionate, hydrocortisone butyrate, hydrocortisone aceponate, hydrocortisone acetate, hydrocortisone buteprate, methylprednisolone aceponate, mometasone furoate, prednicarbate and prednisolone acetate.

Fluid preparations are intended here to mean liquid preparations such as solutions, suspensions, emulsions etc. which, in the case of higher viscosities, may also have a semisolid consistency (e.g. ointments, creams, gels etc.).

The nonaqueous preparations comprise a base of organic solvents or dispersants. A nonaqueous preparation in the sense of this invention may also comprise up to 1% (MN), preferably up to 0.5% (M/V), water, e.g. if the starting materials themselves contain small amounts of water. (“% (M/V)” means mass of the relevant substance in grams per 100 ml of finished preparation.)

The preparations of the invention may comprise protic or aprotic solvents or dispersants or mixtures of both types.

Protic solvents or dispersants which may be mentioned are:

Monohydric or polyhydric alcohols: examples of monohydric alcohols are propanol, isopropanol, ethanol, butanol, isobutanol, 2-hexyldecanol, benzyl alcohol, tetrahydrofurfuryl alcohol and octanol. Examples of polyhydric alcohols are glycerol, diethylene glycol, polyethylene glycol and propylene glycol.

The preparations of the invention preferably comprise aprotic solvents or dispersants. Mention may be made in particular of:

alkanes such as, for example, hexane, paraffin and dioctylcyclohexane

ketones such as, for example, acetone, ethyl methyl ketone and methyl isobutyl ketone

amides such, for example, 2-pyrrolidone and N-methylpyrrolidone

mono-, di- and triglycerides (esters of fatty acids and glycerol) such as, for example, coco caprylates/caprates, glyceryl monolinoleate, glyceryl monooleate, glyceryl ricinoleate, medium-chain triglycerides, cottonseed oil, peanut oil, almond oil, sesame oil, olive oil, sunflower oil, safflower oil, rapeseed oil, glycerol monostearate, glycerol distearate and soya oil.

Esters of fatty acids with monohydric alcohols, such as, for example, 2-octyldodecyl myristate, cetearyl ethylhexanoate, decyl cocoate, decyl oleate, ethyl oleate, isocetyl palmitate, isopropyl myristate, isopropyl palmitate, isostearyl isostearate, octyl palmitate, octyl stearate and oleyl erucate.

Esters of fatty acids and propylene glycol, such as, for example, propylene glycol caprylate/caprate, propylene glycol dipelargonate, propylene glycol laurate and propylene glycol monocaprylate.

Other fatty acid esters such as, for example, dibutyl adipate, dicaprylyl carbonate, diethylhexyl carbonate.

Cyclic carbonates such as, for example, propylene carbonate.

Alkoxylated alcohols (ethers of polyethylene glycol and alcohols) such as, for example, laureth, ceteth, ceteareth, steareth, diethylene glycol monoethyl ether and dipropylene glycol monomethyl ether.

Other ethers such as, for example, dicaprylyl ether and octyldodecanol.

Silicone oils such as, for example, dimethicone and cetyldimethicone.

Particularly preferred preparations of the invention are those in which no protic solvent or dispersant is employed. The acids may be dissolved or suspended in the said solvents. The acids are preferably dissolved in the solvents.

Suitable acids are organic or inorganic acids.

Examples of inorganic acids are hydrochloric acid, sulphuric acid, sulphurous acid and phosphoric acid.

Examples of organic acids are saturated aliphatic monocarboxylic acids having up to 18 carbon atoms, such as, for example, formic acid, acetic acid, propionic acid, butyric acid, lauric acid, palmitic acid, stearic acid; mono- or polyunsaturated aliphatic monocarboxylic acids having up to 18 carbon atoms, such as, for example, oleic acid or sorbic acid; aliphatic hydroxy carboxylic acids having up to 10 carbon atoms such as, for example, citric acid, tartaric acid, lactic acid; dicarboxylic acids such as oxalic, malonic, succinic or adipic acid; keto carboxylic acids such as, for example, oxaloacetic acid; aromatic carboxylic acids such as, for example, benzoic acid or phthalic acid; organic sulphonic acids such as, for example, methanesulphonic acid; cycloaliphatic carboxylic acids such as, for example, ascorbic acid.

The acids are preferably employed in concentrations of from 0.01 to 10% (M/V), preferably 0.05 to 5% (M/V), particularly preferably 0.05 to 1% (M/V).

The formulations may comprise further usual, pharmaceutically acceptable additives and excipients. Examples which may be mentioned are
preservatives such as, for example, phenols (cresols, p-hydroxybenzoic esters such as methylparaben, propylparaben etc.), aliphatic alcohols (benzyl alcohol, ethanol, butanol etc.), quarternary ammonium compounds (benzalkonium chloride, cetylpyridinium chloride).
antioxidants such as, for example, sulphites (Na sulphite, Na metabisulphite), organic sulphides (cystine, cysteine, cysteamine, methionine, thioglycerol, thioglycolic acid, thiolactic acid), phenols (tocopherols as well as vitamin E and vitamin E TPGS (d-alpha-tocopheryl polyethylene glycol 1000 succinate), butylated hydroxyanisol, butylated hydroxytoluene, gallic acid derivatives (propyl, octyl and dodecyl gallates).
wetting agents or emulsifiers such as, for example, fatty acid salts, fatty alkyl sulphates, fatty alkylsulphonates, linear alkylbenzenesulphonates, fatty alkyl polyethylene glycol ether sulphates, fatty alkyl polyethylene glycol ethers, alkylphenol polyethylene glycol ethers, alkylpolyglycosides, fatty acid N-methylglucamides, polysorbates, sorbitan fatty acid esters, lecithins and poloxamers.
pharmaceutically acceptable colourings such as, for example, iron oxides, carotenoids, etc.
spreading agents which can be employed are inter alia hexyldodecanol, decyl oleate, dibutyl adipate, dimethicone, glyceryl ricinoleate, octyldodecanol, octyl stearate, propylene glycol dipelargonate and preferably isopropyl myristate or isopropyl palmitate.
penetration enhancers (or permeation enhancers) improve the transdermal administration of medicaments and are known in principle in the prior art (see, for example, chapter 6 of Dermatopharmazie, Wissenschaftliche Verlagsgesellschaft mbH Stuttgart, 2001). Examples which may be mentioned are spreading oils such as isopropyl myristate, dipropylene glycol pelargonate, silicone oils and their copolymers with polyethers, fatty acid esters (e.g. oleyl oleate), triglycerides, fatty alcohols and linolene. DMSO, N-methylpyrrolidone, 2-pyrrolidone, dipropylene glycol monomethyl ether, octyldodecanol, coleyl macrogol glycerides or propylene glycol laurate can likewise be used.

The medicaments of the invention are generally suitable for use in humans and animals. They are preferably employed in animal management and animal breeding among agricultural and breeding livestock, zoo, laboratory and experimental animals, and pets, specifically and in particular among mammals.

The agricultural and breeding livestock include mammals such as, for example, cattle, horses, sheep, pigs, goats, camels, water buffalos, donkeys, rabbits, fallow deer, reindeer, fur-bearing animals such as, for example, mink, chinchilla, racoon, and birds such as, for example, chickens, geese, turkeys, ducks, pigeons and ostriches. Examples of preferred agricultural livestock are cattle, sheep, pigs and chickens.

Laboratory and experimental animals include dogs, cats, rabbits and rodents such as mice, rats, guinea-pigs and golden hamsters.

Pets include dogs, cats, horses, rabbits, rodents such as golden hamsters, guinea-pigs, mice, also reptiles, amphibians and birds for keeping at home and in zoos.

The preparations of the invention can in principle be administered in all usual ways, e.g. parenterally, orally, or, in particular, topically (e.g. dermally).

### EXAMPLES

### Example 1

0.05 g of dexamethasone 21-acetate, 0.5 g of clotrimazole and X g of acid (see below) are dissolved in 931 g of medium-chain triglycerides (Miglyol 812). 0.114 g of pradofloxacin trihydrate and 1.8 g of colloidal silicon dioxide (Aerosil 200) are dispersed therein with vigorous stirring. The suspension is subsequently homogenized using a rotor-stator.

Example 1a: 0.1 g of sorbic acid

Example 1b: 0.2 g of sorbic acid

Example 1c: 0.5 g of sorbic acid

Example 1d: 0.1 g of stearic acid

Example 1e: 0.2 g of stearic acid

Example 1f: 0.5 g of stearic acid

Example 1g: 0.1 g of propionic acid

Example 1h: 0.2 g of propionic acid

Example 1i: 0.5 g of propionic acid

The stability of the dexamethasone acetate was investigated by storing at 25° C., 40° C. and 50° C. for 6 weeks. FIG. 1 shows that the formation of the degradation product dexamethasone could be reduced concentration-dependently by the acids used.

### Example 2

0.1 g of betamethasone 21-valerate and 0.2 g of propionic acid are dissolved in 940 g of propylene glycol caprylate/caprate (Miglyol 840). 2.0 g of hydrophobic colloidal silicon dioxide (Aerosil R 974) are dispersed therein with vigorous stirring. The suspension is then homogenized using a rotor-stator. The result is a colourless, slightly turbid liquid.

### Example 3

0.5 g of hydrocortisone acetate and 0.5 g of stearic acid are dissolved in 850 g of isopropanol. The result is a colourless clear liquid.

### FIGURES

FIG. 1: Degradation of dexamethasone acetate to dexamethasone in Examples 1a-1f of the invention after storage for 6 weeks

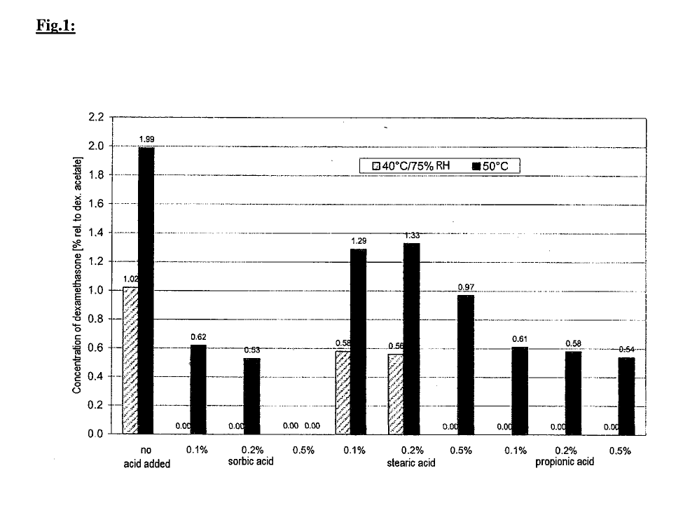

1.-10. (canceled)

11. A method of stabilizing a nonaqueous fluid pharmaceutical preparation comprising at least one glucocorticoid ester, the method comprising contacting the glucocorticoid ester with at least one acid selected from the group consisting of formic acid, acetic acid, propionic acid, butyric acid, lauric acid, palmitic acid, stearic acid, oleic acid, sorbic acid, citric acid, oxaloacetic acid, tartaric acid, methanesulphonic acid, lactic acid and ascorbic acid, and wherein preparation comprises no protic solvents or dispersants.

12. The method of claim 11, wherein the concentration of the acid in the preparation is between 0.01 and 10%.

13. The method of claim 11, wherein the acid is sorbic acid.

14. The method of claim 11, wherein the acid is stearic acid.

15. The method of claim 11, wherein the acid is propionic acid.

16. The method of claim 11, wherein the glucocorticoid ester is esterified at C17 or C21.

17. The method of claim 11, wherein the glucocorticoid ester is dexamethasone acetate or betamethasone valerate.
